(19) United States
(12) Patent Application Publication
(10) Pub. No.: US 2009/0232870 A1
(43) Pub. Date: Sep. 17, 2009

(54) APPARATUS AND METHOD OF RETAINING AND RELEASING MOLECULES FROM NANOSTRUCTURES BY AN EXTERNAL STIMULUS

(75) Inventors: Sunil SRIVASTAVA, Oak Brook, IL (US); Arjan QUIST, Schaumburg, IL (US)

Correspondence Address: ROCKEY, DEPKE & LYONS, LLC, SEARS TOWER, SUITE 5450, CHICAGO, IL, 60606-6306, US

(73) Assignee: RICHMOND CHEMICAL CORPORATION, Oak Brook, IL (US)

(21) Appl. No.: 12/403,222

(22) Filed: Mar. 12, 2009

Related U.S. Application Data
(60) Provisional application No. 61/069,281, filed on Mar. 13, 2008.
(60) Provisional application No. 61/131,795, filed on Jun. 12, 2008.

(51) Int. Cl. A61K 9/00; A61K 38/28

(52) U.S. Cl. 424/423; 514/3; 977/906

(57) ABSTRACT

An apparatus and method for using nanostructures, such as nanopores, nanofibers, nanowells, or nanocones as carriers for drugs, biomarkers and/or biomolecules. The apparatus and method for use on implant surfaces to retain and release drugs, biomarkers and/or biomolecules on command by an external stimulus.

### CROSS-REFERENCE TO RELATED APPLICATIONS

This application claims priority to U.S. Provisional Patent Application Ser. No. 61/069,281 filed Mar. 13, 2008 and U.S. Provisional Patent Application Ser. No. 61/131,795 filed Jun. 12, 2008, the entire disclosures of which are incorporated herein by reference. Priority to this application is claimed under 35 U.S.C. §§119 and/or 120.

### BACKGROUND OF THE INVENTION

1. Field of the Invention

The present invention is generally related to the use of nanostructures as carriers for molecules. More particularly, the present invention can be used on implant surfaces to retain and release drugs, biomarkers and/or biomolecules on command by an external stimulus.

2. Description of the Prior Art

Nanopores are known in the art for the purpose of sensing macromolecules. They have been applied as stochastic sensors for biological molecules, and can identify and quantify analytes based on nanopore current conductance. For example, biomolecules are electrophoretically driven to a nanopore which is effective in determining the concentration and size distribution of particles. Nanopores can be prepared using different types of technologies such as, but not limited to, organic membrane proteins or by synthetic methods. The advantage of the latter is that the pore size can be tailored.

Techniques known in the art to produce synthetic nanopores include, but are not limited to, ion beam sculpting, micromolding, latent track etching, electron beam based technologies, chemical etching of alloys, semiconductor surfaces, or ceramic compounds and nanotubes. Such nanotubes can be silicon based, carbon based, and metal oxide based. Carbon nanotubes can be produced on catalyst particles using plasma enhanced chemical vapor deposition or plasma spraying techniques. Once a carbon nanotube array is created, it can itself function as a template to form metal oxide nanotubes and nanofibers. To achieve this, a metal can be deposited over the carbon nanotube, followed by subsequent oxidation to form a metal oxide, and finally removal of the carbon tube template by a burning process, leading to the production of hollow metal oxide nanofibers.

Biomaterial implant devices are also known in the art and are frequently used in applications relating to artificial hips, elbows, knees, pacemakers, intraocular lenses, heart valves, and coronary stents. In the United States close to 500,000 patients have hip or knee replacements each year. The material used for such implants are bone grafts, metals, polymers, ceramics and composites. Composites consist mostly of bioinert material with a bioactive material such as hydroxyapatite or bioglass. The standard for long term implantation success of bone implants is a complete osseointegration. Orthopedic and dental implants are commonly coated with titanium oxide coatings because of its excellent biocompatibility and superior mechanical properties. It is known that an implant surface coated with nanostructured features, such as carbon nanotubes, improve bone cell growth. Particularly, an electrochemical anodic oxidation of titanium or aluminum leads to improved characteristics. Such anodization processes can be adjusted to produce nanoscale tubular structures of titanium oxide. Calcium phosphates such as hydroxyapatite, which are the main inorganic component of bone, have particle sizes of 20-40 nanometer, and integrate well with such nanostructured titanium oxide having features in the order of 40 to 100 nanometers.

Another area commonly known for their use of implant devices is in the field of cardiology. In cardiology, stents are placed into coronary arteries that may have narrowed or been blocked by heart disease. Often, such stents are coated with immunosuppressive and antiproliferative drugs that are slowly released into the arteries' bloodstream. Such procedures of stent placement are performed nearly 1,000,000 times annually, with a mean cost of $44,000 per procedure, including around $3,000 for the stent itself (2005 data, American Heart Association). Generally, in the case of drug-eluting stents, a polymer coating is used as a drug reservoir and drug delivery regulating layer. Such drug eluting stents coated with, for instance paclitaxel or sirolimus, reduce the rate of restenosis and prevents the need for repeat procedures in patients with coronary artery disease. However, several recurrent problems are present with the current uses of such polymer coatings in stents, as well as other polymer based implants, such as inflammatory reactions, the need for a common solvent for drugs and polymers, polymer fracture during expansion, and delayed endothelium growth. Currently, certain stents use a titanium oxide layer or other ceramic layer for drug elution.

Other medical applications of the present invention include a number of organ implants/transplants with a nanostructured retention and release surface either in, at the surface of, or nearby the implant. It is also contemplated that the present invention has applications in the field of nanofiltration, nanosieves, and other filtrations using hybrid organic-inorganic, nanoporous materials, for solvent drying or use as a molecular sieve, where the control of opening and closing the nanostructures may be useful to adjust filter properties on demand. In this case, the nanostructures will not need a molecular payload, but the invention will merely trigger the open or closed state of the pore system.

Currently available drug eluting coatings such as polymers and nanostructure surfaces are used in a way that does not allow for precise active control of drug release, but merely releases the drug from the moment of incorporation into the body over a period of time depending on the type of surface, the structure of the surface, the concentrations of reagent used, and other properties. Consequently, there is a need for controlled retention and release of molecules from coatings of stents, as well as bone replacements for joints, dental implants or other implants, in order to provide safe and effective treatments for implant patients. The present invention provides a nanosurface or nanostructure, capable of being used with implants, in order to actively control the retention and/or release of molecules by an external stimulus, such as a radio-frequency field, magnetic field, electric field, infrared/thermal or other electromagnetic field in order to provide customized drug treatments and therapies to patients. The present invention is provided to overcome limitations and drawbacks of the prior art and to provide novel aspects not heretofore available.

### SUMMARY OF THE INVENTION

The present invention is directed to an apparatus and method of releasing molecules in a controlled manner into tissue surrounding the site of an implant material in a body. The present invention provides for retention and release of drugs, biomarkers and/or biomolecules on command directly from a biocompatible nanosurface by modifying the nanostructures used on the outer layer of the implant.

One aspect of the present invention provides a nanosurface having at least one nanostructure that is capable of retaining and releasing a molecule based on an external stimulus.

Another aspect of the present invention provides an apparatus for releasing molecules directly from an implant. The apparatus comprises an implant having at least one nanostructure for facilitating the retention and release of a molecule based on an external stimulus.

In yet another aspect of the present invention, the apparatus has a first surface and a second surface. The first surface is an implant. The second surface is contiguous to the first surface and covers a portion of the implant. The second surface has at least one nanostructure for facilitating the retention or release of a biomolecule based on an external stimulus.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1 is a schematic of an apparatus of the present invention;

FIGS. 2A-2C is a schematic demonstrating opening and closing of a nanostructure of the present invention;

FIGS. 3A-3B is a schematic demonstrating opening and closing of a nanostructure via a magnetic mechanism of the present invention;

FIG. 4A-4B is a schematic demonstrating a method of altering the shape of the nanostructure of the present invention; and

FIG. 5 is a schematic depicting an alternate embodiment of the present invention.

### DETAILED DESCRIPTION

The present invention is capable of embodiments in many different forms. Preferred embodiments of the invention are disclosed with the understanding that the present disclosure is to be considered as exemplifications of the principles of the invention and are not intended to limit the broad aspects of the invention to the embodiments illustrated.

The present invention is directed to an apparatus and method of retaining and releasing molecules in a controlled manner into tissue surrounding the site of an implant in a body. Current implants are limited to drug release from a surface immediately after implantation in the body. Such known releases are performed passively using drugs embedded in polymer layers or drugs embedded in the top layer of the implants, such as titanium oxide or hydroxyapatite nanostructures. The present invention allows for active retention and release of molecules on command directly from a biocompatible nanosurface by modification of the nanostructures used within the implant or on an outer surface of the implant. The present invention further provides for delayed or slow release of such molecules by varying release rates of the nanostructures in different areas of the release apparatus.

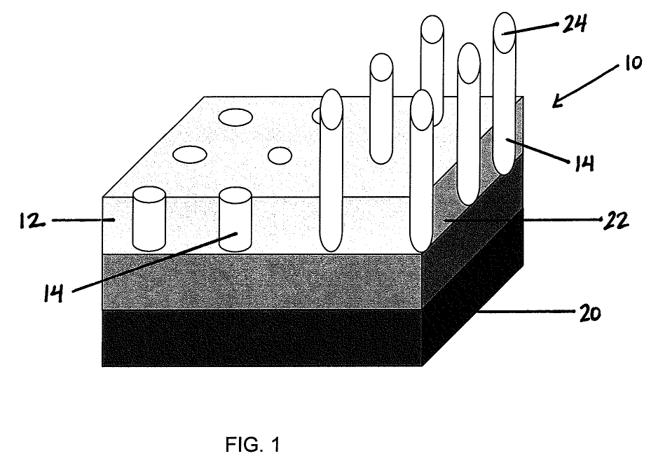

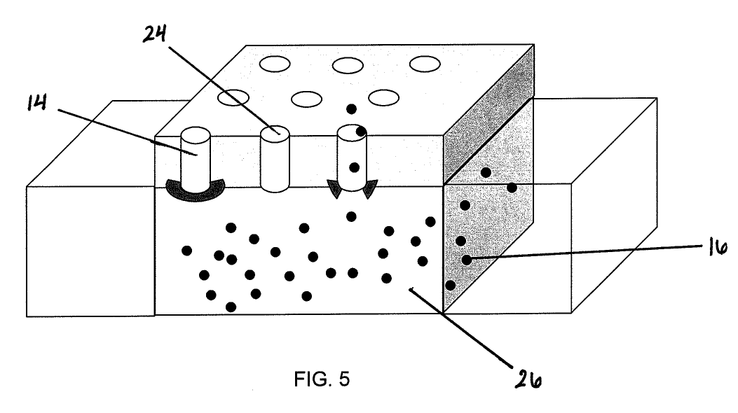

FIGS. 1-5 illustrate the release apparatus and method of using the present invention. As shown in FIGS. 1 and 5, in one embodiment the release apparatus 10 comprises a nanosurface 12 having at least one nanostructure 14 for retaining and releasing a molecule 16 based on an external stimulus 18 (not shown). In one embodiment, a first surface 20 and a second surface 22 are provided. The first surface 20 is an implant such as, but not limited to, a joint implant, a dental implant, a stent, or a vascular implant. The second surface 22 is contiguous to the first surface 20 and covers a portion of the implant. The second surface 22 has at least one nanostructure 14 for retaining or releasing a molecule 18, which is described in greater detail below. The nanostructure 14 functions to trigger the retention and release of biomolecules. The nanostructure 14 has an opening 24 that terminates to the exterior of the apparatus 10. Although one embodiment of the present invention teaches a two surface apparatus, it is contemplated that the apparatus may comprise multiple surfaces. Multiple surfaces may be beneficial for storage of larger amounts of drug molecules in a middle layer, while a nanosurface on top of the storage layer is used for the controlled release of such molecules. Furthermore, multiple surfaces may be used where one intermediate surface holds the drug molecules in nanopores, nano-capillaries or nanowells, while a top nanosurface is used to form a bottleneck structure that can be triggered in an open or closed state. In yet another embodiment, the nanostructures 14 are integrated directly into the implant itself for retaining and releasing drugs, biomarkers and/or biomolecules in a controlled manner into the tissue surrounding the site of the implant in the body. The configuration of the nanostructure is described in detail below.

The present invention is directed to using nanosurfaces or nanostructures with implants such as, but not limited to, joint implants, dental implants, stents or vascular implants. The implant is generally constructed from, but is not limited to, stainless steel, carbon, titanium oxide, hydroxyapatite, metal oxides or ceramic materials. As shown in FIG. 5, a reservoir 26 may be provided within the implant, or within a surface contiguous to the implant, for housing the molecule being retained or released. As discussed above, in one embodiment, a second surface 22 having at least one nanostructure 14 is provided to cover or coat a portion of the implant. The second surface 22 may be constructed, but is not limited to, titanium oxide or other metal oxides. As shown in FIG. 1, additional layers may be added to the second surface, such as a layer of hydroxyapatite or other bone growth promoting material, to improve biocompatibility and bone growth.

The present invention provides for nanostructures that retain and release molecules based on an external stimulus. These nanostructures are located either directly in the implant or in another surface, such as a nanosurface, covering a portion of the implant material. The nanostructures can consist of various configurations capable of retaining and releasing molecules including, but not limited to, nanopores, nanowells, nanotubes or nanocones. The nanostructure may be made of silicon or other semiconductors, carbon, metal oxides such as titanium or aluminum oxide, stainless steel or ceramic materials. The nanostructures are constructed in the nanoporous surface using techniques known in the art such as lithography, ion beam sculpting, micromolding, latent track etching, electron beam based technologies, chemical etching of alloys, semiconductor surfaces, or ceramic compounds and nanotubes.

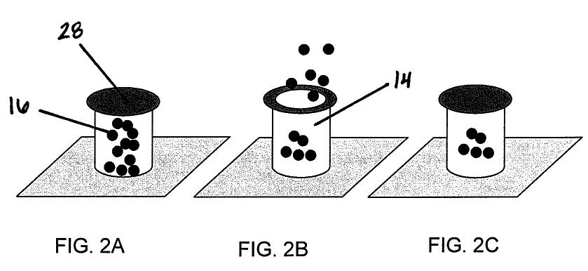

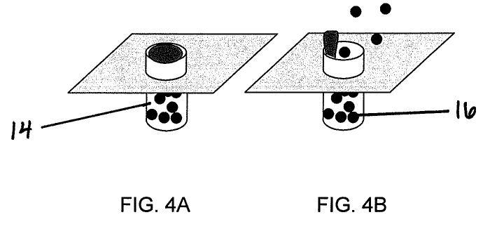

As shown in FIGS. 2A-4B, the present invention contemplates numerous possible retention and release mechanisms. One such mechanism includes capping the outer pore layers, fibers or wells of the nanostructures with an obstruction 28, after absorption/intake of the molecule to be retained and released, illustrated in FIGS. 2A-2C. The nanostructure obstruction may be made of material comprising silicon, semiconductor material, magnetic particles, polymer particles, protein or other biomolecules. Other possible obstructions include different electroactive molecular species capable of rearranging themselves differently depending on the vector direction of applied electric fields based on their different oxidation states. Microarray coatings of different electroactive species could be achieved by micro-inkjet based or dip pen probe technologies, providing areas on the implant that can be opened at different times and for different time periods. Alternatively, obstructing the outer pore of the nanostructure can be performed with semiconducting material, actuated material, carbon based structures, or structures consisting of molecular compounds. As shown in FIGS. 4A-4B, the nanostructure may incorporate a larger particle in the nanopore, nanowell or nanofiber that provides for a delayed or slow release of drugs, biomarkers and/or biomolecules by varying the release rates from different areas of the apparatus.

The nanosurface can be loaded with molecules, such as but not limited to drugs, biomarkers, biomolecules, proteins, polymers, peptide and/or polysaccharides. More specifically, one polysaccharide that can be used with the present invention is inulin, a prebiotic having a beneficial effect on bone metabolism and bone health, by enhancing calcium absorption and bone density. Additionally various drugs may be used including, but not limited to, pro-healing drugs such as dexamethasone, anti-proliferation drugs such as paclitaxel and sirolimus, immunosuppressant drugs or any combination of these drugs may be used with the present invention.

As described above, in one embodiment of the present invention bone growth stimulating drugs may be incorporated in hydroxyapatite coatings on top of a titanium oxide surface. Similarly, nanoporous or nanofibrous titanium oxide structures can be used as drug reservoirs that slowly release a drug into the tissue surrounding the implant. This may be achieved by dissolving the drug or biomarker of interest in a solvent and allowing the nanoporous titanium oxide film to soak up the dissolved biomarker. These nanostructured films can be produced by mixing a titanium chloride precursor with a block copolymer, applying it to a surface, and subsequently aging at high temperatures and calcinations.

As discussed above, the nanostructures may be employed to guide the biomolecules and molecular compounds stored inside the nanosurface or underneath the nanosurface. As shown in FIGS. 2A-4B, the present invention discloses a design that allows for opening and closing of nanopores, nanowells, or nanofibers by means of an external stimulus such as a radio-frequency field (RF field), magnetic field, infrared field, thermal field, electromagnetic field, optical stimulus or other physical stimulus. It is understood that such an external stimulus may be applied in a physician's office. Preferably, a handheld (or other) device may be used to provide a local RF field, magnetic field or infrared field. Activating such a handheld device in the vicinity of the implant, but outside the patient's body, would induce a response in the nanostructure. For RF fields, a particular frequency and/or amplitude may be used to trigger the response of the nanostructures, thus releasing molecules, or stop the release of molecules. Different frequency ranges could be used for triggering separate areas on the same implanted device, each having different molecular contents for molecular variation, or the same molecule for dose variations. Similarly, magnetic fields of different strengths may be used to trigger or stop molecule release when magnetic restriction structures are use to block nanostructures filled with molecules. Different field strengths may be used to trigger different areas for molecule or dose variations. Infrared optical fields may be used as an alternative, in which the infrared radiation penetrates the tissue and can trigger molecular compounds, such as those used in hinge parts of capped nanostructures.

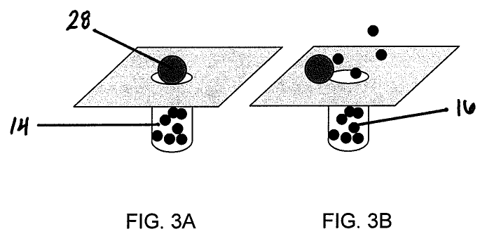

Alternative embodiments of the present invention employ magnetic nano- or micrometer sized particles that are linked to the nanostructure mouth edges by a chemical linker, as illustrated in FIGS. 3A-3B. For example, magnetic particles are available that consist of an iron oxide magnetic core, shielded by a polymer coating that can be tailored with chemical termination groups such as amino, carboxyl, or thiol groups. Similarly, the end opening of carbon nanotubes can also be modified by similar reactive groups. A spacer may be linked in between the nanostructures opening and the magnetic particle to create a reversible pore valve. The magnetic particles can be pulled out of the pore opening by a magnetic trigger thus retaining or releasing trapped molecules from the nanostructures surface into the surrounding tissue. The external trigger leads to enconversion of chemical groups on molecules attached near the opening of these synthetic nanopores.

In an alternative embodiment, the nanostructures are closed by binding or incorporating a larger particle, polymer, biomolecule or protein to the nanopore/nanofiber/nanowell opening, as shown in FIGS. 4A-4B. This particle may contain or be bound to a magnetic particle, semiconductor or metal oxide structure, biomolecular or other structure that can be moved, or deformed by a magnetic field, RF field, or other physical force field. Deformation of the particle, for example stretching of a polymer by pulling a magnetic particle bound to the polymer, or dislocation of the particle, releases the compounds trapped in or underneath the nanopore, nanowell, or nanofiber.

In another embodiment, as shown in FIG. 5, compounds of interest, such as biomolecules, protein, polymers, peptides and polysaccharides, may be stored in a small reservoir that is covered by a porous membrane, and embedded into the implant. The implant itself may be covered by nanostructures, such as but not limited to, silicon, carbon, ceramic, metal oxide, and more specifically titanium oxide. Such nanostructures may be closed on the reservoir side of the porous membrane, by magnetic particles, silicon or other semiconductor structures that can be activated by magnetic field, RF field, infrared/thermal or other electromagnetic fields. This construction prevents particles from leaving the reservoir eliminating any toxic effects from the valve operating mechanism into the surrounding tissue. Such particles and structures are capable of performing in a reversible manner.

In another embodiment, the invention will allow for loading of multiple drugs, biomarkers, polysaccharides, peptides and other molecular compounds onto a stent (cardiac stent, or other stent or other implant device), and release the molecular compounds: sequentially in a time-controlled manner, one-by-one on demand, as a combined release of two or more compounds simultaneously, simultaneously or subsequently at different release rates. This is achieved by triggering only a select area of capped nanostructures to open by designing different regions of capped nanopore structures that respond to different trigger signals, such as but not limited to magnetic fields of different strengths, and/or by created bottleneck caps on the pores that allow for different release rates from different areas on the device.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the following claims.

What is claimed is:

1. A nanosurface having at least one nanostructure for retaining and releasing a molecule based on an external stimulus.

2. The nanosurface of claim 1, wherein the nanostructure comprises biocompatible materials comprising carbon, titanium oxide, hydroxyapatite, metal oxides, stainless steel or ceramic materials.

3. The nanosurface of claim 1, wherein the nanostructure is capped with an obstruction.

4. The nanosurface of claim 1, wherein the nanostructure comprises nanotubes, nanocones, nanopores, or nanowells.

5. The nanosurface of claim 1, wherein the nanostructure is formed by lithography, ion beam sculpting, micromolding, latent track etching, electron beam based technology, chemical etching of alloys, semiconductor surfaces, or ceramic compounds and nanotubes.

6. The nanosurface of claim 1, wherein the molecule comprises drugs, biomarkers, biomolecules, proteins, polymers, peptides and polysaccharides.

7. The nanosurface of claim 1, wherein the molecule comprises inulin, pro-healing drugs, anti-proliferation drugs, or immunosuppressant drugs.

8. The nanosurface of claim 1, wherein the molecule comprises combinatorial drug release.

9. The nanosurface of claim 1, wherein the external stimulus comprises a magnetic field, radio-frequency field, infrared/thermal or electromagnetic field.

10. The nanosurface of claim 1, wherein the nanostructure is capable of reversibly opening and closing based on the external stimulus.

11. The nanosurface of claim 3, wherein the external stimulus triggers removal of the obstruction blocking an opening of the nanostructure.

12. The nanosurface of claim 3, wherein the obstruction is made of a material comprising silicon, semiconductor material, magnetic particles, polymer particles, protein, or biomolecules.

13. The nanosurface of claim 1, wherein the nanostructure has a valve capable of being deformed by the external stimulus to trigger the retention or release of molecules.

14. The nanosurface of claim 1, wherein the nanostructure is triggered independently by different external stimuli, by a same external stimulus, by stimuli having varying strengths, or by stimuli having different magnetic fields.

15. The nanosurface of claim 1, wherein the nanostructure is capable of being preloaded with two or more different types of molecules.

16. The nanosurface of claim 1, wherein the external stimulus provides for sequential release of the molecules, or different types of molecules.

17. An apparatus for retaining and releasing a molecule comprising:
an implant having at least one nanostructure for retaining and releasing a molecule based on an external stimulus.

18. The apparatus of claim 17, wherein the implant comprises a joint implant, a dental implant, a stent or a vascular implant.

19. The apparatus of claim 17, wherein the implant comprises stainless steel, carbon, titanium oxide, hydroxyapatite, metal oxides or ceramic materials.

20. The apparatus of claim 17, wherein the molecule is released into a tissue surrounding the implant.

21. The apparatus of claim 20, further comprising a reversible valve system allowing for the flow of trapped molecules from a nanostructure into the tissue surrounding the implant.

22. An apparatus for retaining and releasing a molecule comprising:
a first surface comprising an implant;
a second surface contiguous to the first surface, the second surface having at least one nanostructure for retaining or releasing a molecule based on an external stimulus.

23. The apparatus of claim 22, wherein the second surface comprises a titanium oxide or other metal oxide.

24. The apparatus of claim 22, further comprising a reservoir housing the molecule in the first surface.
